(19) United States
(12) Patent Application Publication
(10) Pub. No.: US 2016/0193390 A1
(43) Pub. Date: Jul. 7, 2016

(54) Perivascular Delivery System And Method

(71) Applicant: Wisconsin Alumni Research Foundation, Madison, WI (US)

(72) Inventors: Lian-Wang Guo, Madison, WI (US); Kenneth Craig Kent, Fitchburg, WI (US); William L. Murphy, Waunakee, WI (US); Xiaohua Yu, Mansfield Center, CT (US)

(21) Appl. No.: 14/987,038

(22) Filed: Jan. 4, 2016

Related U.S. Application Data
(60) Provisional application No. 62/099,826, filed on Jan. 5, 2015.

(51) Int. Cl. A61L 29/16; A61L 29/06; A61L 29/14; A61M 25/00

(52) U.S. Cl. CPC A61L 29/16; A61M 25/0043; A61L 29/06; A61L 29/148; A61L 29/146; A61M 2025/0057; A61L 2300/416

(57) ABSTRACT

A perivascular delivery system and method are provided for preventing the development of restenosis of a blood vessel. The perivascular delivery system includes a sheath having inner face engageable with an outer surface of the blood vessel and first and second ends. The sheath is fabricated from a bioresorbable polymer. An anti-proliferative drug is loaded into the sheath. The anti-proliferative drug is delivered from the sheath to the blood vessel over time.

### CROSS REFERENCE TO RELATED APPLICATION

This application claims the benefit of U.S. provisional patent application Ser. No. 62/099,826 filed Jan. 5, 2015, the entire contents of which is hereby expressly incorporated by reference.

### REFERENCE TO GOVERNMENT GRANT

This invention was made with government support under HL068673, HL093282, and 03016381 awarded by the National Institutes of Health. The government has certain rights in the invention.

### FIELD OF THE INVENTION

This invention relates generally to the treatment of restenosis, and in particular, to a perivascular delivery system and method for preventing the development of restenosis of a blood vessel following vascular intervention.

### BACKGROUND AND SUMMARY OF THE INVENTION

As is known, the thickening of the subintimal layer of a blood vessel is the universal response of a blood vessel to injury. This thickening of the subintimal layer of the blood vessel is known as intimal hyperplasia and leads to restenosis, or the pathological renarrowing of a blood vessel following vascular intervention. Restenosis develops after balloon angioplasty of atherosclerotic lesions, or following open surgical procedures such as bypass or endarterectomy, wherein an injury is inflicted to the vessel wall. Neointimal plaque is typically formed by proliferative vascular smooth muscle cells (SMCs) from the media or myofibroblasts that migrate from the perivascular layers into the neointimal space.

Despite an in depth understanding of this process, as well as, the development of inhibitors, treatments for restenotic disease have lagged because of the lack of an optimal clinical means of drug delivery. Over the past decade substantial clinical progress has been made in the treatment of post-angioplasty restenosis using drug-eluting stents. However, these intravascular delivery systems are not applicable to open surgical procedures, including bypass, endarterectomy and dialysis access. Even drug eluting stents as a method of drug delivery are imperfect in that residual stenosis remains and there is damage to the endothelium and consequential thrombosis. These limitations, as well as the need for options for open surgery, have led to attempts to develop perivascular delivery systems.

It can be appreciated that at the time of open surgery, a vessel is readily accessible, thereby making application of drug to the vessel more direct and easily achievable. On the other hand, there remains a conspicuous lack of clinical options to prevent intimal hyperplasia following open vascular surgeries. A major obstacle is the absence of a viable technique for perivascular local drug delivery. A number of methods have been explored for perivascular delivery of anti-proliferative drugs to reconstructed arteries or veins using a variety of polymers as a vehicle, including drug-releasing polymer gel depots, microspheres, cuffs, wraps/films, or meshes. While each method has its own advantages, none has advanced to clinical trials, likely due to various limitations revealed in animal studies, such as moderate efficacy, lack of biodegradation, or mechanical stress to the blood vessel. Thus, there remains an unmet clinical need for a perivascular delivery system for preventing intimal hyperplasia, and hence restenosis, that is durable yet biodegradable, non-disruptive to the vessel, and can release a drug in a controlled and sustained manner.

Therefore, it is a primary object and feature of the present invention to provide a perivascular deliver system and method for preventing restenosis.

It is a further object and feature of the present invention to provide a perivascular deliver system and method for preventing restenosis that utilizes a polymeric material that is durable and biodegradable.

It is a further object and feature of the present invention to provide a perivascular delivery system and method for preventing restenosis that has the ability to release a desired drug in a controlled and sustained manner.

It is a still further object and feature of the present invention to provide a perivascular delivery system and method for preventing restenosis that is simple to use and inexpensive to manufacture.

In accordance with the present invention, a perivascular delivery system is provided for preventing the development of restenosis of a blood vessel having an outer surface and a circumference. The perivascular delivery system includes a sheath having inner face engageable with the outer surface of the blood vessel and first and second ends. The sheath is fabricated from a bioresorbable polymer. An anti-proliferative drug is loaded into the sheath. The anti-proliferative drug is delivered from the sheath to the blood vessel over time.

The sheath may be porous and/or may include a plurality of perforations therethrough. Further, the sheath has a length between the first and second ends. The length of the sheath is less than the circumference of the blood vessel. The length of the sheath is at least 60% of the circumference of the blood vessel. The bioresorbable polymer may be selected from the group consisting of poly(ε-caprolactone) (PCL), poly(lactic-co-glycolic acid) (PLGA), and poly(lactic acid) (PLLA) or be a blend of one or more of such polymers.

It is contemplated for the anti-proliferative drug to be rapamycin, resveratrol or JQ1 . The anti-proliferative drug delivered from the sheath may have substantially linear drug release kinetics. The anti-proliferative drug being delivered from the sheath has drug release kinetics, the drug release kinetics being dependent upon the bioresorbable polymer of the sheath.

In accordance with a further aspect of the present invention, a method is provided for preventing the development of restenosis of a blood vessel having an outer surface and a circumference. The method includes the steps of positioning a sheath about the circumference of the blood vessel such that an inner face of the sheath engages the outer surface of the blood vessel. A first end of the sheath is spaced from a second end of the sheath such that a portion of the blood vessel is exposed therebetween. An anti-proliferative drug is delivered from the sheath to the blood vessel over time.

The anti-proliferative drug is embedded into the sheath and the sheath is fabricated from a bioresorbable polymer. The bioresorbable polymer may be selected from a group consisting of poly(ε-caprolactone) (PCL), poly(lactic-co-glycolic acid) (PLGA), and poly(lactic acid) (PLLA). Alternatively, the bioresorbable polymer is a blend and the blend may include at least one of poly(ε-caprolactone) (PCL), poly(lactic-co-glycolic acid) (PLGA), and poly(lactic acid) (PLLA). The sheath may be porous and may include a plurality of perforations therethrough.

The anti-proliferative drug may be, e.g. rapamycin, resveratrol or JQ1, and the anti-proliferative drug delivered from the sheath has drug release kinetics. The drug release kinetics are dependent upon the bioresorbable polymer of the sheath. It is contemplated for the anti-proliferative drug to have substantially linear drug release kinetics.

In accordance with a still aspect of the present invention, a method is provided for preventing the development of restenosis of a blood vessel having an outer surface and a circumference. The method includes the steps of embedding the anti-proliferative drug into a sheath. The sheath is fabricated from bioresorbable polymer. The sheath is positioned about the circumference of the blood vessel such that an inner face of the sheath engages the outer surface of the blood vessel. A first end of the sheath is spaced from a second end of the sheath such that a portion of the blood vessel is exposed therebetween. The anti-proliferative drug is delivered from the sheath to the blood vessel over time. The anti-proliferative drug delivered from the sheath has drug release kinetics. The drug release kinetics are dependent upon the bioresorbable polymer of the sheath.

The bioresorbable polymer may be selected from a group consisting of poly(ε-caprolactone) (PCL), poly(lactic-co-glycolic acid) (PLGA), and poly(lactic acid) (PLLA). Alternatively, the bioresorbable polymer may be a blend which includes at least one of poly(ε-caprolactone) (PCL), poly(lactic-co-glycolic acid) (PLGA), and poly(lactic acid) (PLLA). In addition, the sheath may be porous. It is contemplated for the anti-proliferative drug to be rapamycin. The anti-proliferative drug delivered from the sheath has substantially linear drug release kinetics.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is a schematic, isometric view sheath for use in the perivascular delivery system of the present invention;

FIG. 2 is a schematic end view of the sheath of FIG. 1;

FIG. 3 is a schematic view showing the steps for fabricating the sheath of FIG. 1;

FIG. 4 is a schematic view showing the steps for positioning the sheath of FIG. 1 on a blood vessel;

FIG. 5 is a schematic, isometric view, partially in section, showing the sheath of FIG. 1 positioned on a blood vessel;

FIG. 6 is a graphical representation showing the percentage of rapamycin released from sheaths fabricated from various bioresorbable polymers over time;

FIG. 7 is a graphical representation showing the percentage of rapamycin released from sheaths fabricated from various bioresorbable polymers during various predetermined time periods;

FIG. 8 is a graphical representation showing the cumulative percentage of rapamycin released from sheaths fabricated from various blends of bioresorbable polymers over time;

FIG. 9 is a graphical representation showing the percentage of rapamycin released from sheaths fabricated from various blends of bioresorbable polymers during various predetermined time periods;

FIG. 10 is a graphical representation showing the mean intima versus media area ratios of blood vessels treated with control sheaths and with rapamycin-loaded PCL sheaths;

FIG. 11 is a graphical representation showing the mean lumen areas of the blood vessels treated with control sheaths and with rapamycin-loaded PCL sheaths;

FIG. 12 is a graphical representation showing the mean number of Ki67 positive cells in the blood vessels treated with control sheaths and with rapamycin-loaded PCL sheaths; and

FIG. 13 is a graphical representation showing the mean re-endothelialization indices of blood vessels which occurred when treated with control sheaths and with rapamycin-load PCL sheaths.

### DETAILED DESCRIPTION OF THE DRAWINGS

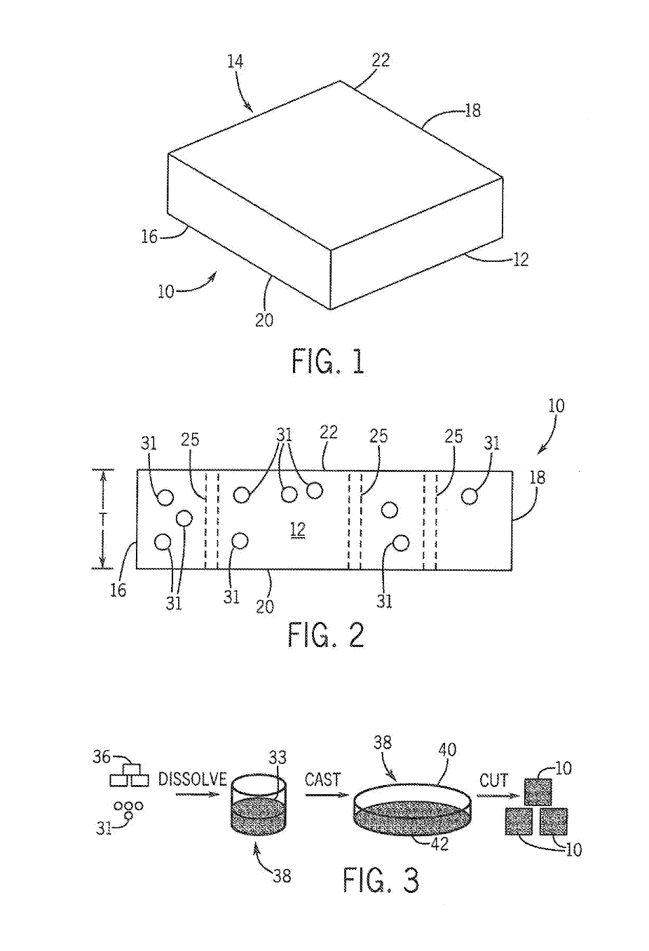

Referring to FIGS. 1 and 2, a perivascular delivery sheath for use in connection with the perivascular delivery system and the methodology of the present invention is generally designated by the reference numeral 10. In the depicted embodiment, sheath 10 has a generally square configuration and includes first and second ends 12 and 14, respectively, and first and second edges 16 and 18, respectively. However, it can be appreciated that sheath 10 may have other configurations without deviating from the scope of the present invention. Sheath 10 is further defined by opposite first and second sides 20 and 22, respectively, separated by a thickness “T”. It is contemplated for the thickness “T” of sheath 10 to be in the range of 20 to 100 micrometers (μm) and preferably to be approximately 50 μm.

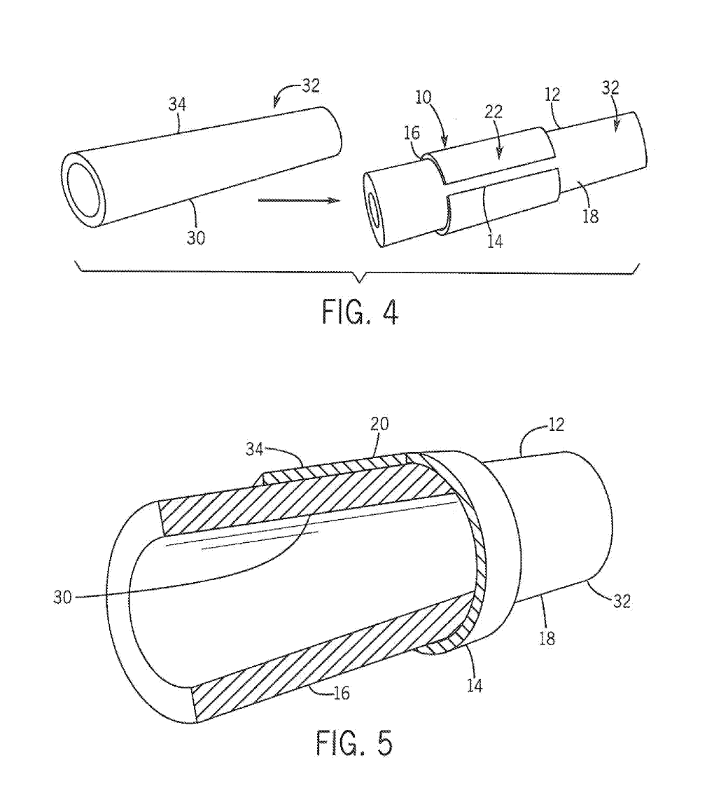

Sheath 10 is fabricated from a bioresorbable polymer loaded with an anti-proliferative drug. The bioresorbable polymer should have sufficient flexibility to prevent constriction of or further damage to injured segment 30 of blood vessel 32, FIGS. 4-5, when in use, as hereinafter described, and have the ability to sustain drug delivery in humans for an extended period of time. It is intended for the bioresorbable polymer to optimize the in vitro release profile of the anti-proliferative drug loaded therein. For example, sheath 10 may be fabricated from poly(ε-caprolactone) (hereinafter referred to as “PCL”), poly(lactic-co-glycolic acid) (hereinafter referred to as “PLGA”), Poly(lactic acid) (hereinafter referred to as “PLLA”) or a blend thereof. However, it is also contemplated to fabricate sheath 10 from other bioresorbable polymers without deviating from the scope of the present invention. Various anti-proliferative drugs may be loaded in the bioresorbable polymer. For example, as hereinafter described, rapamycin, an anti-proliferative drug clinically used in drug-eluting stents, may be loaded in the bioresorbable polymer of sheath 10. However, other anti-proliferative drugs, such as Resveratrol or JQ1, may be loaded in the bioresorbable polymer of sheath 10 without deviating from the scope of the present invention.

In order to fabricate sheath 10, a solvent casting method may be used. Referring to FIG. 3, it is contemplated to dissolve a desired anti-proliferative drug 31 into a solvent 33 to form a solution. A bioresorbable polymer 36 is added to the solution and stirred for a predetermined time period in a darkened environment to form a mixture 38. The mixture 38 is cast in a mold 40 and the mold 40 is inserted into a fume hood (not shown) for a predetermined time period in order for the solvent 34 to evaporate from the mixture 38. The casted mixture 38 defines a film 42, which may be cut into sheaths 10 of predetermined sizes, after polymerization. Sheaths 10 are subsequently vacuum dried overnight in a darkened environment to eliminate any residual solvent 32. While depicted as a solid film of material, it can be appreciated that sheath 10 may include optional perforations 25 or the like to allow fluid communication therethrough, FIG. 2.

Referring to FIGS. 4-5, in operation, once access is provided to blood vessel 32 having injured segment 30, sheath 10 is longitudinally placed onto injured segment 30 of blood vessel 32. First side 20 of sheath 10 is circumferentially wrapped about outer surface 34 of injured segment 30 such that sheath 10 partially surrounds blood vessel 32. First and second ends 12 and 14, respectively, of sheaths 10 are spaced form each other such that sheath 10 covers less than the 100% of the circumference of the blood vessel 32. It is contemplated for sheaths 10 to cover approximately 60% to 100% of the circumference of the blood vessel 32, and preferably, approximately 80-90% of the circumference of blood vessel 32. By partially surrounding blood vessel 32 with sheath 10, dynamic movement of blood vessel 32 is allowed, thereby minimizing the potential damage to blood vessel 32 from sheath 10 during the expansion and contraction thereof. Once sheath 10 is placed onto injured segment 30 of the blood vessel 32, the intrinsic to adhesive quality of sheath 10 is used to retain sheath 10 on the injured segment 30. Alternatively, sheath 10 may be retained in place on injured segment 30 by a suture. Blood vessel 32 is then buried in tissue in the body and any incision made to provide access to blood vessel 32 is closed.

Once positioned on injured segment 30 of blood vessel 32, the anti-proliferative drug is released from sheath 10 and delivered to injured segment 30. It can be appreciated that the perivascular delivery of the anti-proliferative drug is evenly distributed along the entire length of sheath 10. The drug release kinetics and the durability of sheath 10 are dependent on the bioresorbable polymer or the blend of bioresorbable polymers from which sheath 10 is fabricated, as hereinafter described. Preferably, the drug release kinetics of sheath 10 are modulated to a desired pattern, such as the steady and sustainable release of the anti-proliferative drug from sheath 10, and sheath 10 is provided with sufficient durability to sustain drug delivery in humans for an extended period of time, e.g. 90 days or more.

In order to evaluate the efficacy, experiments were conducted to determine the release rate of the anti-proliferative drug from sheath 10 in vitro and to determine if sheath 10 infused with the desired anti-proliferative drug would be effective for inhibiting restenosis in a rat balloon angioplasty model. In accordance with such experiments, sheaths 10 were fabricated, as heretofore described, by infusing various bioresorbable polymers (PLGA, PLLA, or PCL) with rapamycin, an anti-proliferative drug proven to be effective for inhibiting restenosis in rats. In addition, sheaths 10 were prepared using the same procedures, but with no rapamycin added.

Sheaths 10 were fabricated by dissolving 10 milligrams (mg) of rapamycin in 2.2 milliliters (ml) of chloroform to form a solution. A volume, e.g. 220 mg, of a bioresorbable polymer (PLGA, PLLA, or PCL), is added to the rapamycin/chloroform solution and stirred in a darkened environment for approximately 30 minutes. The polymer/rapamycin/chloroform mixture is cast in a 60 millimeter (mm), polytetrafluoroethylene (hereinafter referred to as “PTFE”) dish and inserted into a fume hood (not shown) for approximately 48 hours to evaporate the chloroform. Preferably, the film of the polymer/rapamycin/chloroform mixture in the PTFE dish has a thickness in the range of 20 and 100 μm. The thickness of the film of polymer/rapamycin/chloroform mixture may be controlled by varying the amount of polymer added into the PTFE dish. To produce sufficient mechanical flexibility necessary for use as a perivascular sheath, the polymer films were prepared with an average thickness of around 50 μm.

The casted mixture or film is cut into sheets of a desired size, e.g. (1 centimeter (cm)×1 cm) or (1 cm×0.5 cm), and subsequently vacuum dried overnight in a darkened environment to eliminate any residual chloroform. Thereafter, the rapamycin-loaded polymeric sheaths were stored at −20° C. until use. As fabricated, sheath 10 (1 cm−0.5 cm) includes approximately 100 μg of rapamycin, which is in the range of concentrations proven to be effective for inhibiting restenosis in the rat balloon angioplasty model.

In order to efficiently screen the sheaths 10 fabricated from each of the bioresorbable polymers (PLGA, PLLA, or PCL), an in vitro system was used to evaluate their rapamycin release kinetics. In a 0.6 milliliter(ml) microcentrifuge tube, sheaths 10 fabricated from each of the bioresorbable polymers (PLGA, PLLA, or PCL) and loaded with rapamycin were incubated in a 500 microliter (μl) release medium of phosphate buffered saline (PBS) buffer (pH 7.4) including 0.02% NaN3 and 10% isopropyl alcohol (IPA), which was included to inhibit rapamycin degradation. At predetermined intervals, 200 μl of the release medium was replaced with an equal volume of fresh release medium and the former was transferred into a UV-free 96-well plate. The concentrations of rapamycin in the release mediums in the well plate were measured by determining the absorbance at 278 nanometers (nm) using a microplate reader for a time period of 50 days. A calibration standard curve was prepared in the same release medium and used to calculate the amount of released rapamycin.

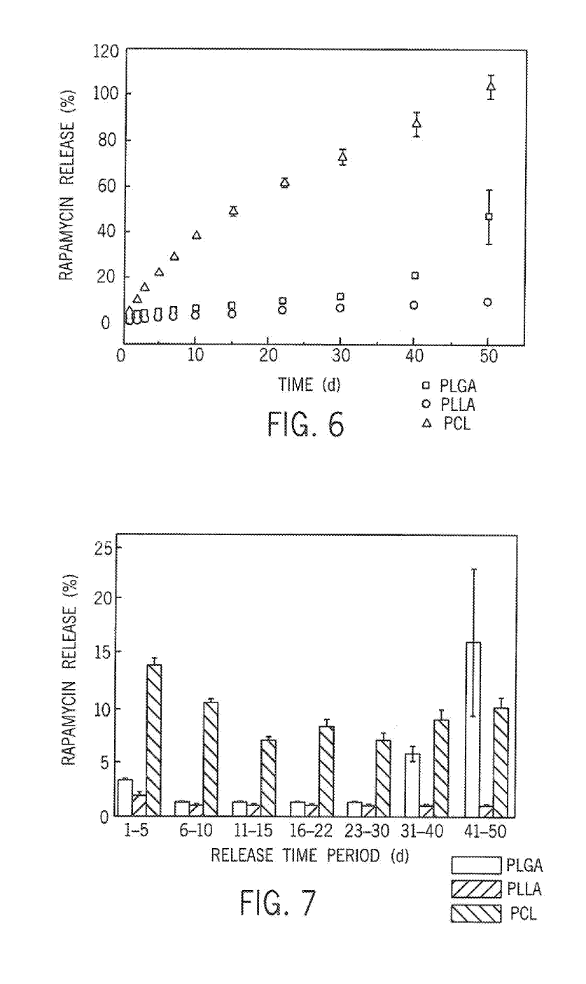

Utilizing the in vitro system heretofore described, it was found that the choice of the bioresorbable polymers (PLGA, PLLA, or PCL) had a dominant effect on the release kinetics of the rapamycin from sheaths 10. More specifically, referring to FIGS. 6-7, it was found that the release rate of rapamycin from a PLGA sheath was sustained over an initial portion of the time period, e.g. the first 30 days, and then followed by accelerated release rate in a subsequent portion of the time period, e.g. the last 20 days. On the other hand, the PLLA sheath provided very slow release of rapamycin throughout the 50 day time period. The PCL sheath produced a faster, near-linear release of rapamycin over the 50 day time period. It was found that after 50 days of release, 10% and 46% of rapamycin were released from the PLLA sheath and the PLGA sheath, respectively, whereas nearly 100% rapamycin was released from the PCL sheath within the same time frame. Analysis of the daily release revealed a minor initial burst of rapamycin from all 3 bioresorbable polymers (PLGA, PLLA, or PCL) over the first 10 days of the time period, although the PCL, sheath showed faster release compared to the other two during this period, FIG. 7.

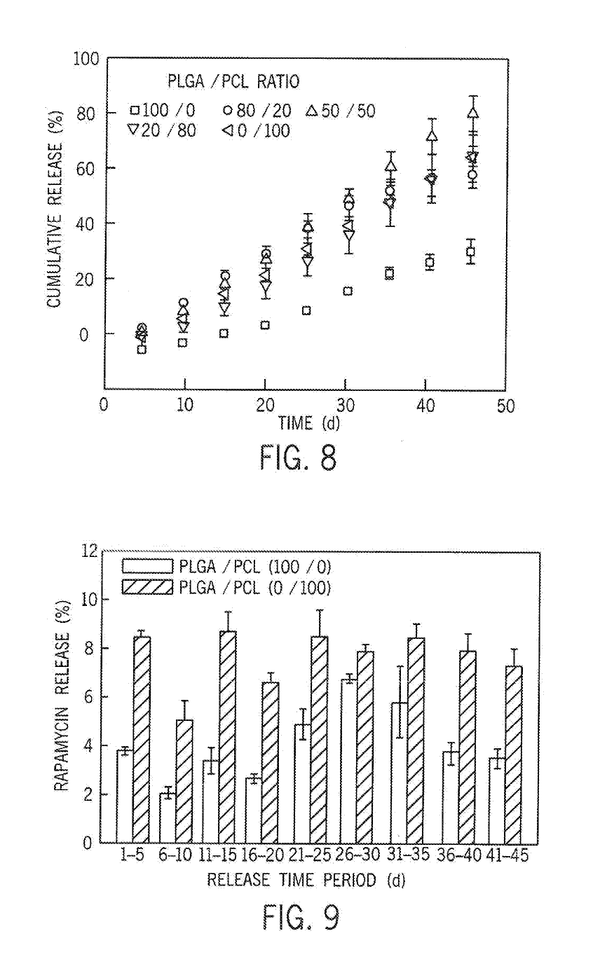

To refine the release kinetics of the rapamycin from sheath 10, it is contemplated to fabricate sheath 10 from a blend of bioresorbable polymers (PLGA, PLLA, or PCL). By way of example, a series of sheaths 10 were fabricated utilizing blends of PLGA/PCL in different ratios, FIGS. 8-9. It can be appreciated that by blending different ratios of PCL into PLGA, the rapamycin release kinetics of the PLGA/PCL sheath may be modified to substantially mirror the rapamycin release kinetics of a sheath fabricated from pure PCL. Hence, by manipulating the bioresorbable polymer composition of sheath 10, the drug release kinetics of sheath 10 may be modulated to a desired pattern, such as the steady and sustainable release of the anti-proliferative drug from sheath 10.

In order to further evaluate the efficacy, sheaths 10, infused with the desired anti-proliferative drug, e.g. rapamycin, were implanted in rats to determine if the sheaths 10 would be effective for inhibiting restenosis in the rat balloon angioplasty model. More specifically, the rats were anesthetized, and a Fogarty arterial embolectomy catheter was inserted into the left common carotid artery via an arteriotomy in the external carotid artery. The animals used in the experiment were from the same litter of rats. To produce arterial injury, a balloon was inflated and withdrawn to the carotid bifurcation for a predetermined number of times, e.g. three. The external carotid artery was then permanently ligated, and blood flow was resumed.

Sheaths 10 (1 cm×0.5 cm) fabricated from each of the bioresorbable polymers (PLGA, PLLA, or PCL) and loaded with rapamycin were longitudinally placed onto injured segments, approximately 1.5 cm, of the common carotid arteries of the rats and wrapped about the injured segments such that sheaths 10 partially surrounded carotid arteries, FIGS. 4-5. First and second ends 12 and 14, respectively, of sheaths 10 were spaced from each other such that sheaths 10 covered less than the 100% of the circumferences of the carotid arteries. It is contemplated for sheaths 10 to cover approximately 60% to 100% of the circumferences of the carotid arteries, and prefrerably, approximately 80-90% of the circumferences of the carotid arteries. Once the sheaths 10 were placed onto the injured segments of the carotid arteries of the rats, the neck incisions were closed using sutures and the rats were kept on a 37° C. warm pad for recovery. In addition to the sheaths 10 fabricated from the bioresorbable polymers (PLGA, PLLA, or PCL) and loaded with rapamycin applied to the injured carotid arteries of the rats, as heretofore described, sheaths 10 fabricated from the bioresorbable polymers (PLGA, PLLA, or PCL) without rapamycin (hereinafter referred to collectively as the “control sheaths”) were also applied to the injured carotid arteries of rats.

Two weeks after the balloon injury, the balloon-injured artery segments treated with the control sheaths and the sheaths 10 fabricated from the bioresorbable polymers (PLGA, PLLA, or PCL) loaded with rapamycin were collected from the same parts of carotid arteries in the rats. The two week time period is a time point that represents the most rapid neointima accumulation after injury. The collected segments were fixed in paraffin sections having a selected thickness (e.g. 5 μm) and excised at equally spaced intervals to form sections for examination. Thereafter, the excised sections were stained with hematoxylin-eosin (H&E) for morphometric analysis. The areas enclosed respectively by the external elastic lamina (EEL) and the internal elastic lamina (IEL) and lumen area were measured. Intimal area (IEL area minus lumen area) and medial area (EEL area minus IEL area) were then calculated. Intimal hyperplasia was assessed for each section with the area ratio of intima versus media, FIG. 10. For each of these parameters, data from all the sections from a given segment were pooled to generate a mean for each rat. The means from all the rats treated with the sheaths having the same construction were averaged, and the standard error of the mean (SE) was calculated.

It is initially noted that thrombosis was rare in the twelve rats treated with sheaths 10 fabricated from PCL. More specifically, thrombosis was produced in only two out of twelve rats treated with sheaths 10 fabricated from PCL. In addition, among the twelve rats treated with sheaths 10 fabricated from PCL, ten of the treated rats (4 treated with control sheaths and 6 treated with sheaths loaded with rapamycin) were without apparent pathology (thrombosis, infection, or scarring). On the other hand, sheaths 10 fabricated from either PLLA or PLGA produced frequent arterial thrombosis in the treat rats. It is noted that 2 out of the 4 rats treated with PLGA sheaths and 12 out of 14 animals treated with PLLA sheaths developed thrombotic occlusion in the treated carotid arteries. This drastic difference between PCL and the other two polymers underscores the influence of physical properties of polymer drug carriers on the outcomes of their perivascular application.

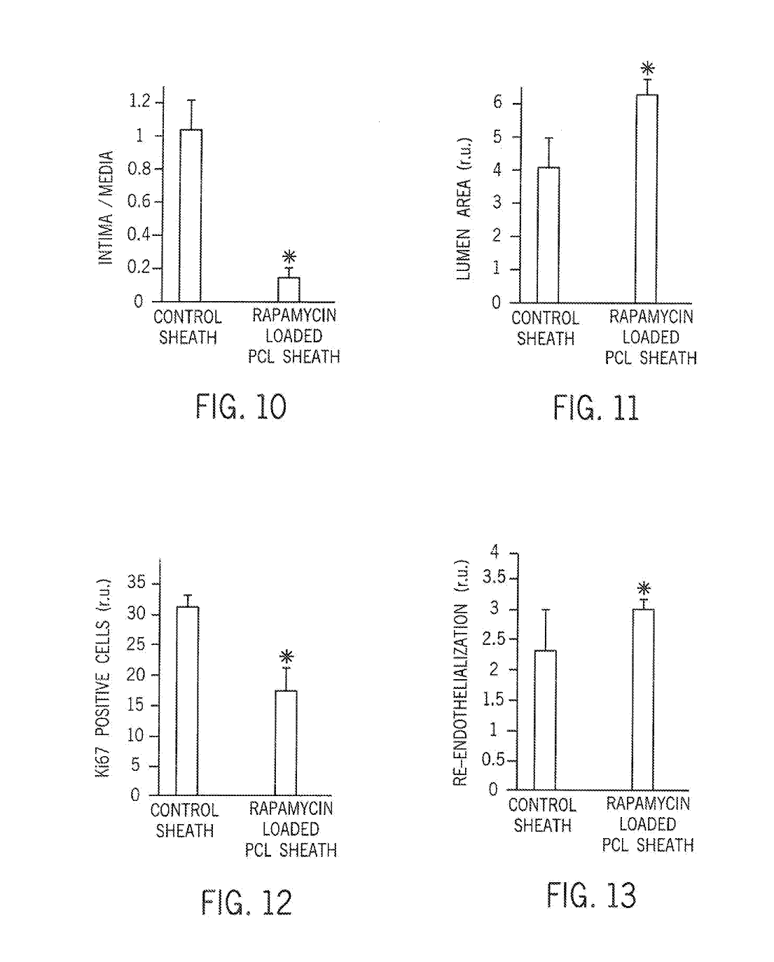

Further, it was found that the sheaths 10 fabricated from PCL and loaded with rapamycin produced a dramatic inhibitory effect on intimal hyperplasia (85% reduction) in the carotid arteries of the treated rats, without the side effect of endothelial damage. As a result, the lumen area was increased by 155%, FIG. 11. As such, it can be appreciated that the efficacy of the rapamycin-loaded PCL sheath 10 constitutes a significant improvement over prior perivascular delivery systems. In addition, ki67-positive (proliferative) cells were significantly reduced by more than 40% in the medial and neointimal layers in the carotid arteries treated with the rapamycin-loaded PCL sheaths 10, as compared to the carotid arteries treated with the PCL control sheaths, FIG. 12. Since an established function of rapamycin is the inhibition of SMC proliferation and migration, the data indicates that the rapamycin-loaded PCL sheaths 10 effectively delivered the rapamycin into SMCs in the vessel wall to mitigate the growth of neointimal plaque.

Shrinkage of the vessel wall, or constrictive remodeling, is often an important contributor to the loss of lumen size in addition to intimal hyperplasia. It is noted that no constrictive remodeling of the carotid arteries was seen in the rats treated with the PCL sheaths 10. Further, the recovery of the endothelium in the carotid arteries in the rats treated with the PCL sheaths 10 fourteen days after the denudation caused by the balloon injury was not impaired by the rapamycin delivered from the perivascular PCL sheaths 10, FIG. 13. It is further noted that the PCL sheaths 10 used to treat the rats remained intact at least for 90 days, while the subcutaneously embedded PLGA and PLLA sheaths were partially dissolved at 15 days and 90 days, respectively. The excellent durability of the PCL sheath is a desired feature for sustained drug delivery in humans, where nonregressive intimal plaque develops for up to two years after reconstructive surgery.

Finally, it is noted that only roughly 20% of the rapamycin loaded in the PCL sheaths was released fourteen days after being placed in the rats. However, 20% of the rapamycin in the PCL sheaths generated a profound inhibitory effect on neointima. Further, more than 30% of rapamycin still remained in the PCL sheaths after 45 days. Hence, it can be appreciated that the inhibitory effect of the rapamycin-loaded PCL sheath 10 on neointimal hyperplasia will extend for periods well beyond 45 days.

As described, a perivascular delivery system is provided that dramatically reduces neointima without showing side effects of either endothelial damage or constrictive remodeling. The excellent efficacy of the perivascular delivery system of the present invention incorporates appropriate physical properties suitable for normal vessel wall physiology; sustained, nearly linear drug release kinetics; perivascular drug delivery evenly spread along an injured segment of a blood vessel; and excellent durability (at least 3 months in vivo).

Various modes of carrying out the invention are contemplated as being within the scope of the following claims particularly pointing and distinctly claiming the subject matter that is regarded as the invention.

We claim:

1. A perivascular delivery system for preventing the development of restenosis of a blood vessel having an outer surface and a circumference, comprising:
a sheath having inner face engageable with the outer surface of the blood vessel and first and second ends, the sheath fabricated from a bioresorbable polymer; and
an anti-proliferative drug loaded into the sheath, the anti-proliferative drug being delivered from the sheath to the blood vessel over time.

2. The perivascular delivery system of claim 1 wherein:
the sheath has a length between the first and second ends; and
the length of the sheath is less than the circumference of the blood vessel.

3. The perivascular delivery system of claim 2 wherein the length of the sheath is at least 60% of the circumference of the blood vessel.

4. The perivascular delivery system of claim 1 wherein the bioresorbable polymer is selected from the group consisting of poly(ε-caprolactone) (PCL), poly(lactic-co-glycolic acid) (PLGA), and poly(lactic acid) (PLLA).

5. The perivascular delivery system of claim 1 wherein the bioresorbable polymer includes at least one of poly(ε-caprolactone) (PCL), poly(lactic-co-glycolic acid) (PLGA), and poly(lactic acid) (PLLA).

6. The perivascular delivery system of claim 1 wherein the anti-proliferative drug is one of rapamycin, resveratrol and JQ1.

7. The perivascular delivery system of claim 1 wherein the sheath is porous.

8. The perivascular delivery system of claim 1 wherein the sheath includes a plurality of perforations therethrough.

9. The perivascular delivery system of claim 1 wherein the anti-proliferative drug being delivered from the sheath has substantially linear drug release kinetics.

10. The perivascular delivery system of claim 1 wherein the anti-proliferative drug being delivered from the sheath has drug release kinetics, the drug release kinetics being dependent upon the bioresorbable polymer of the sheath.

11. A method for preventing the development of restenosis of a blood vessel having an outer surface and a circumference, comprising:
positioning a sheath about the circumference of the blood vessel such that an inner face of the sheath engages the outer surface of the blood vessel;
spacing a first end of the sheath from a second end of the sheath such that a portion of the blood vessel is exposed therebetween; and
delivering an anti-proliferative drug from the sheath to the blood vessel over time.

12. The method of claim 11 comprising the additional step of embedding the anti-proliferative drug into the sheath.

13. The method of claim 11 wherein the sheath is fabricated from a bioresorbable polymer, the bioresorbable polymer selected from a group consisting of poly(ε-caprolactone) (PCL), poly(lactic-co-glycolic acid) (PLGA), and poly(lactic acid) (PLLA).

14. The method of claim 11 wherein the sheath is fabricated from a bioresorbable polymer, wherein:
the bioresorbable polymer is a blend; and
the blend includes at least one of poly(ε-caprolactone) (PCL), poly(lactic-co-glycolic acid) (PLGA), and poly(lactic acid) (PLLA).

15. The method of claim 11 wherein the anti-proliferative drug is one of rapamycin, resveratrol and JQ1.

16. The method of claim 11 wherein the sheath is porous.

17. The method of claim 11 wherein the sheath includes a plurality of perforations therethrough.

18. The method of claim 11 wherein the anti-proliferative drug delivered from the sheath has substantially linear drug release kinetics.

19. The method of claim 11 wherein the anti-proliferative drug delivered from the sheath has drug release kinetics, the drug release kinetics being dependent upon the bioresorbable polymer of the sheath.

20. A method for preventing the development of restenosis of a blood vessel having an outer surface and a circumference, comprising:
embedding the anti-proliferative drug into a sheath, the sheath fabricated from bioresorbable polymer;
positioning the sheath about the circumference of the blood vessel such that an inner face of the sheath engages the outer surface of the blood vessel;
spacing a first end of the sheath from a second end of the sheath such that a portion of the blood vessel is exposed therebetween; and
delivering the anti-proliferative drug from the sheath to the blood vessel over time; wherein the anti-proliferative drug delivered from the sheath has drug release kinetics, the drug release kinetics being dependent upon the bioresorbable polymer of the sheath.

21. The method of claim 20 wherein the bioresorbable polymer selected from a group consisting of poly(ε-caprolactone) (PCL), poly(lactic-co-glycolic acid) (PLGA), and poly(lactic acid) (PLLA).

22. The method of claim 20 wherein:
the bioresorbable polymer is a blend; and
the blend includes at least one of poly(ε-caprolactone) (PCL), poly(lactic-co-glycolic acid) (PLGA), and poly(lactic acid) (PLLA).

23. The method of claim 20 wherein the anti-proliferative drug is one of rapamycin, resveratrol and JQ1.

24. The method of claim 20 wherein the sheath is porous.

25. The method of claim 20 wherein the anti-proliferative drug delivered from the sheath has substantially linear drug release kinetics.
